(19) United States
(12) Patent Application Publication
(10) Pub. No.: US 2018/0250087 A1
(43) Pub. Date: Sep. 6, 2018

(54) SYSTEM AND METHOD FOR MOTION CAPTURE AND CONTROLLING A ROBOTIC TOOL

(71) Applicants: Frank Grasser, Eggolsheim (DE); Holger Mönnich, Friedberg (DE)

(72) Inventors: Frank Grasser, Eggolsheim (DE); Holger Mönnich, Friedberg (DE)

(21) Appl. No.: 15/911,915

(22) Filed: Mar. 5, 2018

(30) Foreign Application Priority Data
Mar. 6, 2017 (EP) 17159352.8

(51) Int. Cl. A61B 34/00; B25J 19/02; A61B 8/00; A61B 6/00

(52) U.S. Cl. CPC A61B 34/70; B25J 19/026; A61B 6/5247; A61B 8/4254; A61B 8/4209

(57) ABSTRACT

System and methods for controlling a robotic medical tool are provided. The system includes the robotic medical tool, a motion capture device, and a controller. The motion capture device is configured to capture a motion of an object. The controller is configured to adjust the robotic tool relative to the object as a function of the captured motion.

### CROSS REFERENCE TO RELATED APPLICATIONS

This application claims the benefit of EP17159352, filed on Mar. 6, 2017, which is hereby incorporated by reference in its entirety.

### FIELD

Embodiments relate to a system and a method for motion capture and for controlling a robotic medical tool.

### BACKGROUND

For precise manipulation of an object or interaction with an object using a robotic medical tool it is necessary to control the robotic medical tool with equal precision. Known systems and methods make use of external optical tracking systems including a camera and physical markers. The markers are physically attached to the object and may be tracked with the camera via direct line of sight and may thus be used to track the motion or movement of the object. The use of physical markers as well as the requirement of a direct line of sight between the markers and the camera are often limiting and undesirable.

### SUMMARY

Embodiments facilitate improved control of a robotic tool.

The scope of the present invention is defined solely by the appended claims and is not affected to any degree by the statements within this summary. The present embodiments may obviate one or more of the drawbacks or limitations in the related art.

A system according to an embodiment includes a robotic medical tool, a motion capture device, and a control unit. The motion capture device is configured to capture a motion or movement of an object, while the control unit is configured to adjust—and control—the robotic tool in dependence of the captured motion or movement.

As used herein, “motion” and “movement” may refer to a motion of the whole object or a motion of only a part of the object.

As used herein, a robotic tool may refer to a device or machine configured to carry out a complex series of actions automatically. The actions may include movement and positioning of the robotic tool or a part thereof in three dimensions, mechanically guiding other devices or an operator, manipulation of and/or interaction with the tracked object, and others. The robotic tool may be computer programmable to provide for specifying in advance the actions to carry out.

The robotic tool may also be described as a medical robot. The robotic tool may include a tool head that is or includes the part of the robotic tool that is used to manipulate the object or interact with the object. The tool head may be changeable to provide for greater flexibility and use cases of the robotic tool. Examples of tool heads may include probes and drills.

The control unit is connected to the robotic tool as well as to the ultrasound device via respective data links. The data links are used to send data representing the captured motion from the ultrasound device to the control unit and to send corresponding control signals or commands from the control unit to the robotic tool. The data links may also be bidirectional to provide a two-way exchange of data, for example providing a feedback control system. The control unit may be configured to control the ultrasound device, for example to automatically follow a movement of the robotic tool. The motion of the specific part of the object closest to the robotic tool and/or containing a point or area of interaction between the robotic tool and the object may be always automatically captured providing for a precise adjustment of the robotic tool relative to the object. The control unit may include at least one microcontroller, microprocessor, storage medium, and/or interface to carry out the described actions.

The ultrasound device may include an ultrasound head or probe. The ultrasound head may be placed in contact with the object to be tracked. For example, tracking of movements of a patient for example during spine surgery. Patient movements may be precisely captured and tracked. The robotic tool may be adjusted or controlled accordingly to compensate the patient movements. Bone material of the patient may be detected using the ultrasound device, for example the spine or a specific vertebra. The movement of bone of the patient may hence be captured and tracked. The captured and/or tracked movement—or corresponding data—is processed to generate corresponding control signals for the robotic tool. Through the control signals the robotic tool may be kept in an intended position relative to the patient or, e.g., relative to the individual bone detected by the ultrasound device.

The ultrasound device may be operated at different ultrasound frequencies. Different frequencies may be suited for capturing and/or tracking different objects and/or different motions and may thus be selected case by case. As an example, an ultrasound device using frequencies between 2 and 15 MHz may provide a lateral resolution between 3 and 0.4 mm and an axial resolution between 0.8 and 0.15 mm

Embodiments may forgo any additional markers on the object and thereby may for example obviate the need for additional incisions in the patient or other object, e.g. for minimally invasive procedures, avoiding unnecessary injury of a patient or damage to an object and improving a workflow. By not relying on an optical camera system to capture and track the motion of the object, embodiments also avoid occlusion problems where the motion of the object is not captured due to another object or person blocking direct line of sight between the camera and the tracked object or marker. Embodiments therefore provides improved flexibility and reliability in capturing and/or tracking the motion of the object.

Embodiments are not limited to patients as objects or medical procedures but may also be applied to manipulations of any object the motion that may be detected with the ultrasound device. For example, objects that are at least partially penetrable by ultrasound and include areas or parts of different density that respond different to ultrasound.

In an embodiment, the system is configured to determine a main plane and/or direction of the motion. The ultrasound device is configured to capture the motion of the object at least in that plane and/or direction. The system and the ultrasound device may be arranged to capture the directional motion of the object with for example the biggest deflection, amplitude, and/or path length. As an example, a vertebra of a patient may substantially only move back and forth in one direction due to a respiratory movement of the patient. The plane or direction may be determined by an operator or may be determined automatically. For example, the plane or direction may be determined dynamically during operation of the system. To determine the plane or direction automatically, the object may be scanned or captured from different sides or angles, e.g. the ultrasound device may be set to observe the object from different points of view. If, for example, the object follows a periodic trajectory, the main plane or direction of movement may be determined by observing the object from different angles over a period of time on the order of the periodic time. Capturing the motion of the object in the main plane or direction of movement may provide for sufficient capturing precision even with a very simple ultrasound device. The object may, for example, be secured in such a way that the object's movement is limited to only one plane or direction. Only one plane or direction of movement may be important or relevant to the outcome or success of the procedure performed by or with the robotic tool.

In an embodiment, the motion capture device is configured to capture the motion of the object in three dimensions. A motion captures device is provided where the motion of the object may be captured regardless of the direction of movement. Precise adjustment of the robotic tool is provided. Flexibility and applicability may be increased as the motion captured device may be used in scenarios where the object and/or the robotic tool moves in a complex pattern. To capture the three-dimensional motion of the object the ultrasound device may include a 3D-ultrasound head or probe.

In an embodiment the ultrasound device includes at least two ultrasound heads. The two ultrasound heads or probes may be arranged in the same housing to effectively form a compact and simple to use 3D-ultrasound device. The two ultrasound heads may also be spaced apart from each other and may, for example, be attached to different mounts or bearings to provide independent positioning of two ultrasound heads. Each ultrasound head may be built very small and compact while at the same time providing for capture of the motion of the object in three dimensions. The two ultrasound heads may be arranged or positioned very flexibly, enabling the use of the system in a wide variety of situations. The two ultrasound heads may also be used to capture the motions of different parts of the object, that may provide a more sophisticated and precise adjustment of the robotic tool relative to the object.

In an embodiment, the system is configured to scan the object and to register the medical tool to a coordinate system of the scan. Embodiments provide that a frame of reference and a position of the object in that frame of reference is established and the robotic tool is aligned or synchronized to that frame of reference allowing for absolute positioning of different components such as the robotic tool, the ultrasound device, and others with respect to the object independently of each other. If for example the robotic tool is moved out of a coverage range or coverage area of the ultrasound device the alignment and knowledge about the precise relative positioning is not automatically lost. To support the functionality any motion of the robotic tool and/or the ultrasound device may be tracked using a motorized bearing, mount, and/or control of the robotic tool and/or the ultrasound respectively.

In an embodiment, the system is configured to scan the object using the ultrasound device. The ultrasound device may be moved along or around the object to scan the object and establish a frame of reference and a position of the object within that frame of reference providing for registration of the different system components that may be moved independently of each other to one another without the need for any further equipment.

In an embodiment, the system is configured to scan the object with an X-ray apparatus. Through the scan a frame of reference and a position of the object within that frame of reference may be established. The robotic tool and the X-ray apparatus may be registered to one another. The X-ray apparatus may provide more precise and/or different data to allow for a precise initial positioning of the robotic tool relative to the scanned object. The ultrasound device may be used to capture and/or track the motion or movements of the object without exposing the object to unnecessary high doses of X-ray radiation. The ultrasound device may also be registered to the X-ray apparatus and the robotic tool or the same frame of reference as the X-ray apparatus and the robotic tool. The X-ray apparatus may be part of the system or part of a separate device.

In an embodiment, the ultrasound device is arranged so that a main detection direction of the ultrasound device extends substantially perpendicular to a central axis of a cone of operation of the robotic tool. The ultrasound device may be arranged on a different side of the object than the robotic tool, e.g. lateral to the object. The arraignment avoids contact or conflict between the robotic tool and the ultrasound device and keeps the point or region of contact or interaction between the robotic tool and the object free for other tools and/or for observation or intervention by an operator. An embodiment provides that the ultrasound device does not have to be positioned next to the robotic tool or even on the same side of the object as the robotic tool since the ultrasound may capture the motion of the whole object or a part of the object, for example, an inner part that is not readily visible from the outside, from different directions. The ultrasound device may be arraigned or positioned opposite the robotic tool. The main detection direction of the ultrasound device may extend substantially antiparallel to the central axis of the cone of operation of the robotic tool. The cone of operation of the robotic tool is defined by a substantially cone-shaped volume extending outward from a point of contact or interaction between the robotic tool and the object. The robotic tool may be moved or positioned within the cone of operation while maintaining its point of contact or interaction with the object.

In an embodiment, at least one ultrasound head of the ultrasound device is integrated within the robotic tool. The ultrasound head may be part of a tool head that is itself part of the robotic tool or attached thereto. The ultrasound head or probe may be disposed to a side or next to the tool head. The ultrasound head does not have be enclosed by a housing of the robotic tool but may instead be attached to the robotic tool or the housing of the robotic tool from the outside. In either case, the ultrasound head is rigidly connected to the robotic tool so the ultrasound head and robotic tool move together or in tandem allowing for the ultrasound device to be aligned with an axis of the robotic tool and/or always captures the motion of exactly the part of the object that is manipulated by or with the robotic tool. The ultrasound head integrated with or attached to the robotic tool may be a single or dual, for example, a 3D-ultrasound head. Depending on the situation or conditions, the robotic tool or a sophisticated motion capturing may have a compact outline.

In addition to the ultrasound head integrated with the robotic tool the system may include at least one additional ultrasound head. The additional ultrasound head may be arranged independently of the robotic tool and may provide an additional angle of motion capture.

A method is provided for controlling a robotic medical tool. As part of the method a motion or movement of an object is captured, and the robotic tool is adjusted relative to the object in dependence of the captured motion. The motion of the object is captured by an ultrasound device. The method may be carried out by or with a system according to the above described embodiments.

### BRIEF DESCRIPTION OF THE FIGURES

FIG. 1 depicts a schematic perspective view of a system engaged in manipulating a patient according to an embodiment.

### DETAILED DESCRIPTION

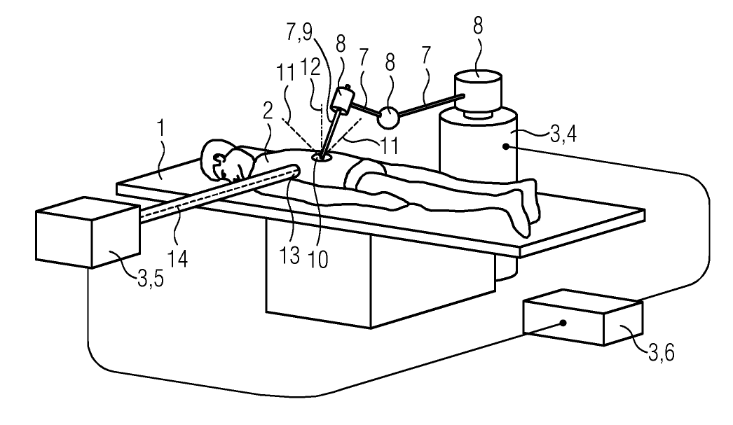

FIG. 1 depicts a schematic view of an operating table 1 on which a patient 2 is positioned. FIG. 1 further depicts a system 3 including a robotic tool 4, an ultrasound device 5, and a control unit 6. The control unit 6 is configured to at least partially control the robotic tool 4 and the ultrasound device 5 and is connected to the robotic tool 4 as well as to the ultrasound device 5 via respective data links or cables. The control unit 6 may alternatively be part of or integrated in the robotic tool 4 and/or the ultrasound device 5.

The robotic tool 4 includes a member or arm with segments 7. The segments 7 are connected through joints 8. The arrangement of the segments 7 and the joints 8 allows complex movements around at least three axes. The segment 7 furthest from a main body of the robotic tool may be a tool head 9 that may include or carry different kinds of types of tools. The tool head 9 or, e.g. a distal end of the tool head 9, is configured for manipulation of or interaction with objects. The manipulated object is the patient 2 or a part thereof, for example, a region of interest (ROI) 10 of the patient. The ROI 10 may for example be or include a spine or a specific vertebra of the patient 2.

The ROI 10 may be considered as a point of origin of a cone of operation of the robotic tool 4. The cone of operation describes a volume of space in which the tool head 9 may move or be positioned while maintaining its contact with the ROI 10. For illustrative purposes, the cone of operation is indicated by two reference lines 11 and a central axis 12. Depending on the situation the cone of operation may not actually be cone-shaped.

A precise control and positioning of the tool head 9 relative to the patient 2 or the ROI 10 is used in many applications of the robotic tool 4. For example, in robot assisted spine surgery, where the robotic tool 4 supports a surgeon to place screws into a vertebra of the patient 2, the screw path is precisely controlled to achieve the intended purpose and prevent injury of the patient 2. Therefore, any motion or movement of the ROI 10 is detected and captured by the ultrasound device 5.

An ultrasound head 13 of the ultrasound device 5 is placed in contact with the patient 2. The ultrasound device 5 and the ultrasound head 13 are disposed lateral to the patient 2, for example, on a different side of the patient 2 than the ROI 10 and the tool head 9. A main detection direction 14 of the ultrasound device 5 extends substantially perpendicular to the central axis 12 of the cone of operation of the robotic tool 4 or the tool head 9. The ultrasound device 5 may, for example, detect bone material of the patient 2, for example, in the ROI 10. The ultrasound device 5 may capture and track a motion of the bone material and by extension of the patient 2.

The captured motion is put in relation to the current position and/or motion of the tool head 9. The robotic tool 4 and the ultrasound device 5 may be registered to one another. An X-ray apparatus (not shown) may be registered to the same frame of reference too.

The motion of the patient 2 and/or the ROI 10 captured by the ultrasound device 5, or corresponding sensor data, may be transmitted to the control unit 6. The control unit 6 processes the data received from the ultrasound device 5 and generates corresponding control signals or commands The commands are sent to the robotic tool 4 to position and/or move the tool head 9 accordingly. The robotic tool, for example, the tool head 9, is controlled, e.g. positioned and/or moved, in dependence of the motion of the patient 2 and/or the ROI 10 captured by the ultrasound device 5. The ultrasound provides live adjustments of the robotic tool 4 during operation.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A system for controlling a robotic medical tool, the system comprising:
the robotic medical tool;
a motion capture device configured to capture a motion of an object; and
a controller configured to adjust the robotic medical tool relative to the object based on the captured motion.

2. The system of claim 1, wherein the motion capture device is an ultrasound device.

3. The system of claim 2, wherein the motion capture device is further configured to determine a main plane, a direction, or the main plane and the direction of the motion of the object, and
wherein the motion capture device is arranged to capture the motion of the object in the main plane, the direction, or the main plane and the direction of the motion of the object.

4. The system of claim 2, wherein the motion capture device is configured to capture the motion in three dimensions.

5. The system of claim 2, wherein the ultrasound device comprises at least two ultrasound heads.

6. The system of claim 2, wherein the system is configured to scan the object and to then register the robotic medical tool to a coordinate system of the scan.

7. The system of claim 6, wherein the system is configured to scan the object with the ultrasound device.

8. The system of claim 1, further comprising:
an X-ray apparatus configured to scan the object.

9. The system of claim 2, wherein the motion capture device is arranged so that a main detection direction of the motion capture device extends substantially perpendicular to a central axis of a cone of operation of the medical robotic tool.

10. The system of claim 2, wherein at least one ultrasound head of the ultrasound device is integrated within the robotic tool.

11. A method for controlling a robotic medical tool, the method comprising:
capturing, by an ultrasound device, a motion of an object; and
adjusting, by a controller, the robotic medical tool relative to the object based on the captured motion.

12. The method of claim 11, further comprising:
determining a main plane, a direction, or the main plane and the direction of the motion,
wherein capturing, by the ultrasound device, the motion of the object comprises capturing, by the ultrasound device, the motion of the object in the main plane, the direction, or the main plane and the direction.

13. The method of claim 11, wherein the motion is captured in three dimensions.

14. The method of claim 11, wherein the ultrasound device comprises at least two ultrasound heads.

15. The method of claim 11, further comprising:
scanning, by the ultrasound device, the object; and
registering the robotic medical tool to a coordinate system of the scan.

16. The method of claim 11, wherein the ultrasound device is arranged so that a main detection direction of the ultrasound device extends substantially perpendicular to a central axis of a cone of operation of the robotic medical tool.
